(19) United States
(12) Patent Application Publication
(10) Pub. No.: US 2021/0059240 A1
(43) Pub. Date: Mar. 4, 2021

(54) NOVEL METHOD FOR BLOOD SERUM PROTEIN ACTIVITY PRESERVATION

(71) Applicant: Chung Chin SUN, Taipei City (TW)

(72) Inventors: Chung Chin SUN, Taipei City (TW); Cheng-Yao SU, Taipei City (TW); Shan Shue WANG, Taipei City (TW); Takuya MIYAGAWA, Taipei City (TW)

(73) Assignee: Chung Chin SUN, Taipei City (TW)

(21) Appl. No.: 16/620,162

(22) PCT Filed: Jun. 13, 2017
(86) PCT No.: PCT/CN2017/087999

§ 371 Date: Dec. 6, 2019

(51) Int. Cl. A01N 1/00

(52) U.S. Cl. CPC A01N 1/00

(57) ABSTRACT

A method for blood serum protein activity preservation is provided. The method comprises the steps of mixing blood serum with one or more protectants selected from the group consisting of albumin, triglyceride, glycerol, dextran, propylene glycol, galactose, alginate, and trehalose; and lyophilizing the mixture.

### FIELD OF THE INVENTION

The present invention relates to a method for blood serum protein activity preservation.

### BACKGROUND OF THE INVENTION

Blood often looks relatively simple, but its composition, from a chemical standpoint at least, tends to be somewhat complex. In most cases it has five main components: serum, plasma, clotting factors, lipids and proteins, and blood cells. Serum is a clear, yellowish colored fluid which is part of the blood that does not contain white or red blood cells. It is essentially the most basic and neutral part of blood, not only acts as a fluid backdrop for many of blood's most important functions, namely shuttling minerals, sugars, and fatty acids from one place to the next but also provides an ideal consistency and climate for allowing blood particles free movement. Serum includes all proteins not used in blood clotting (coagulation) and all the electrolytes, antibodies, antigens, hormones, and any extra substances such as drugs and microorganisms. Its neutrality makes it valuable in a number of different medical tests. Researchers have perfected ways of isolating the substance in order to diagnose a range of different conditions and problems. It is sometimes also used to make eye drops for people with tear duct problems since its consistency often closely mimics that of natural tears.

The state of the art of blood serum storage is to frozen the serum quickly within 24 hours after phlebotomy and store it typically as Fresh Frozen Serum (FFS) up to one year. The FFP may be thawed shortly before use. However, such storage method has at least the following drawbacks: (1) serum may not be stored for a long term unless it is frozen as FFS and stored in an ultra-low temperature refrigerator; and (2) the protein activity cannot be well maintained after thawing for use.

### BRIEF SUMMARY OF THE INVENTION

The present invention provides a method for blood serum protein activity preservation, comprising mixing blood serum with one or more protectants selected from the group consisting of albumin, triglyceride, glycerol, dextran, propylene glycol, galactose, alginate, and trehalose.

Preferably, the method comprising mixing blood serum with two or more protectants selected from the group consisting of albumin, triglyceride, glycerol, dextran, propylene glycol, galactose, alginate, and trehalose. According to certain embodiments of the present invention, the two or more protectants are selected from the group consisting of glycerol, alginate, and trehalose.

In certain embodiments of the present invention, the two or more protectants comprise a first protectant of trehalose and a second protectant of glycerol or alginate. According to one embodiment of the present invention, the two or more protectants comprise trehalose and glycerol. In another embodiment, the two or more protectants comprise trehalose and alginate.

According to the present invention, the blood serum may be further mixed, in the mixing step, with one or more protectant selected from the group consisting of dextran, propylene glycol, sucrose, galactose, triglyceride, and a combination thereof.

Methods of the present invention may be used to protect growth factors in the serum from degradation. The growth factors in the serum include but are not limited to PDGF-AB, TGF-β1, and VEGF.

It is to be understood that both the foregoing general description and the following detailed description are exemplary and explanatory only and are not restrictive of the invention.

### BRIEF DESCRIPTION OF THE DRAWINGS

The foregoing summary, as well as the following detailed description of the invention, will be better understood when read in conjunction with the appended drawing. In the drawings:

FIGS. 1A-1C show the growth factor levels in the serum reconstituted from the serum powder using albumin as the protectant. FIG. 1A shows the levels of PDGF-AB, FIG. 1B shows the levels of TGF-β1, and FIG. 1C shows the levels of VEGF. Amount of protectant used based on the volume of the serum: % means % (w/v). Control: serum only. The difference between data shown in the same style of bar (serum reconstituted after the same period (1 hour, 3 months, or 12 months) of time after lyophilization) with different letters is statistically significant (P<0.05).

FIGS. 2A-2C show the growth factor levels in the plasma reconstituted from the serum powder using gelatin as the protectant. FIG. 2A shows the levels of PDGF-AB, FIG. 2B shows the levels of TGF-β1, and FIG. 2C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 3A-3C show the growth factor levels in the serum reconstituted from the serum powder using glycine as the protectant. FIG. 3A shows the levels of PDGF-AB, FIG. 3B shows the levels of TGF-β1, and FIG. 3C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 4A-4C show the growth factor levels in the serum reconstituted from the serum powder using serine as the protectant. FIG. 4A shows the levels of PDGF-AB, FIG. 4B shows the levels of TGF-β1, and FIG. 4C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 5A-5C show the growth factor levels in the serum reconstituted from the serum powder using triglyceride as the protectant. FIG. 5A shows the levels of PDGF-AB, FIG. 5B shows the levels of TGF-β1, and FIG. 5C shows the levels of VEGF. Amount of protectant used based on the volume of the serum: % means % (v/v). Control: serum only. The difference between data shown in the same style of bar (serum reconstituted after the same period of time (1 hour, 3 months, or 12 months) after lyophilization) with different letters is statistically significant (P<0.05).

FIGS. 6A-6C show the growth factor levels in the serum reconstituted from the serum powder using glycerol as the protectant. FIG. 6A shows the levels of PDGF-AB, FIG. 6B shows the levels of TGF-β1, and FIG. 6C shows the levels of VEGF. Amount of protectant used based on the volume of the serum: % means % (v/v). Control: serum only. The difference between data shown in the same style of bar (serum reconstituted after the same period of time (1 hour, 3 months, or 12 months) after lyophilization) with different letters is statistically significant (P<0.05).

FIGS. 7A-7C show the growth factor levels in the serum reconstituted from the serum powder using dextran as the protectant. FIG. 7A shows the levels of PDGF-AB, FIG. 7B shows the levels of TGF-β1, and FIG. 7C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 8A-8C show the growth factor levels in the serum reconstituted from the serum powder using propylene glycol as the protectant. FIG. 8A shows the levels of PDGF-AB, FIG. 8B shows the levels of TGF-β1, and FIG. 8C shows the levels of VEGF. Amount of protectant used based on the volume of the serum: % means % (v/v). Control: serum only. The difference between data shown in the same style of bar (serum reconstituted after the same period of time (1 hour, 3 months, or 12 months) after lyophilization) with different letters is statistically significant (P<0.05).

FIGS. 9A-9C show the growth factor levels in the serum reconstituted from the serum powder using alginate as the protectant. FIG. 9A shows the levels of PDGF-AB, FIG. 9B shows the levels of TGF-β1, and FIG. 9C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 10A-10C show the growth factor levels in the serum reconstituted from the serum powder using ribose as the protectant. FIG. 10A shows the levels of PDGF-AB, FIG. 10B shows the levels of TGF-β1, and FIG. 10C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 11A-11C show the growth factor levels in the serum reconstituted from the serum powder using arabinose as the protectant. FIG. 11A shows the levels of PDGF-AB, FIG. 11B shows the levels of TGF-β1, and FIG. 11C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 12A-12C show the growth factor levels in the serum reconstituted from the serum powder using glucose as the protectant. FIG. 12A shows the levels of PDGF-AB, FIG. 12B shows the levels of TGF-β1, and FIG. 12C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 13A-13C show the growth factor levels in the serum reconstituted from the serum powder using galactose as the protectant. FIG. 13A shows the levels of PDGF-AB, FIG. 13B shows the levels of TGF-β1, and FIG. 13C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 14A-14C show the growth factor levels in the serum reconstituted from the serum powder using sucrose as the protectant. FIG. 14A shows the levels of PDGF-AB, FIG. 14B shows the levels of TGF-β1, and FIG. 14C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 15A-15C show the growth factor levels in the serum reconstituted from the serum powder using trehalose as the protectant. FIG. 15A shows the levels of PDGF-AB, FIG. 15B shows the levels of TGF-β1, and FIG. 15C shows the levels of VEGF. Amount of protectant used based on the volume of the serum: % means % (w/v). Control: serum only. The difference between data shown in the same style of bar (serum reconstituted after the same period of time (1 hour, 3 months, or 12 months) after lyophilization) with different letters is statistically significant (P<0.05).

FIGS. 16A-16C show the growth factor levels in the serum reconstituted from the serum powder using two (2) protectants. FIG. 16A shows the levels of PDGF-AB, FIG. 16B shows the levels of TGF-β1, and FIG. 16C shows the levels of VEGF. Control: serum only. 1: 2% Dextran+2% Glycerol; 2: 2% Dextran+2% Glycine; 3: 2% Dextran+2% Serine; 4: 2% Dextran+2% Sucrose; 5: 2% Dextran+2% Glucose; 6: 2% Dextran+0.2% Arabinose; and 7: 2% Dextran+2% Ribose. Amount of protectant used based on the volume of the serum: % means % (v/v) for glycerol, and means % (w/v) for other protectants. The difference between data shown in the same style of bar (serum reconstituted after the same period of time (1 hour, 3 months, or 12 months) after lyophilization) with different letters is statistically significant (P<0.05).

FIGS. 17A-17C show the growth factor levels in the serum reconstituted from the serum powder using two (2) protectants. FIG. 17A shows the levels of PDGF-AB, FIG. 17B shows the levels of TGF-β1, and FIG. 17C shows the levels of VEGF. Control: serum only. 1: 2% Alginate+2% Glycine; 2: 2% Alginate+2% Trehalose; 3: 2% Alginate+2% Sucrose; 4: 2% Alginate+2% Glucose; 5: 2% Alginate+2% Galactose; 6: 2% Alginate+0.2% Arabinose; and 7: 2% Alginate+2% Ribose. Amount of protectant used based on the volume of the serum: % means % (w/v). The difference between data shown in the same style of bar (serum reconstituted after the same period of time (1 hour, 3 months, or 12 months) after lyophilization) with different letters is statistically significant (P<0.05).

FIGS. 18A-18C show the growth factor levels in the serum reconstituted from the serum powder using two (2) protectants. FIG. 18A shows the levels of PDGF-AB, FIG. 18B shows the levels of TGF-β1, and FIG. 18C shows the levels of VEGF. Control: serum only. 1: 2% Glycerol+0.2% Triglyceride; 2: 2% Glycerol+2% Glycine; 3: 2% Glycerol+2% Serine; 4: 2% Glycerol+2% Trehalose; 5: 2% Glycerol+2% Sucrose; 6: 2% Glycerol+2% Glucose; 7: 2% Glycerol+2% Galactose; 8: 2% Glycerol+0.2% Arabinose; and 9: 2% Glycerol+2% Ribose. Amount of protectant used based on the volume of the serum: means % (v/v) for triglyceride and glycerol, and means % (w/v) for other protectants. The difference between data shown in the same style of bar (serum reconstituted after the same period of time (1 hour, 3 months, or 12 months) after lyophilization) with different letters is statistically significant (P<0.05).

### DETAILED DESCRIPTION OF THE INVENTION

The present invention provides a method for blood serum protein activity preservation, comprising mixing blood serum with one or more protectants selected from the group consisting of albumin, triglyceride, glycerol, dextran, propylene glycol, galactose, alginate, and trehalose.

Preferably, the method comprising mixing blood serum with two or more protectants selected from the group consisting of albumin, triglyceride, glycerol, dextran, propylene glycol, galactose, alginate, and trehalose. According to certain embodiments of the present invention, the two or more protectants selected from the group consisting of glycerol, alginate, and trehalose.

According to the present invention, the protectants may be added in the following amounts: (1) 0.01-10% (v/v) glycerol based on the volume of the serum, preferably 0.1-5% (v/v); (2) 0.01%-10% (w/v) alginate based on the volume of the serum, preferably 0.1-5% (w/v); and (3) 0.01%-10% (w/v) trehalose based on the volume of the serum, preferably 0.1-10% (w/v).

In certain embodiments of the present invention, the two or more protectants comprise a first protectant of trehalose and a second protectant of glycerol or alginate.

According to one embodiment of the present invention, the two or more protectants comprise trehalose and glycerol. For example, the following amounts of protectants may be added (based on the volume of serum): about 2% (w/v) trehalose, and about 2% (v/v) glycerol.

In another embodiment, the two or more protectants comprise trehalose and alginate. For example, the following amounts of protectants may be added (based on the volume of serum): about 2% (w/v) trehalose, and about 2% (w/v) alginate.

According to the present invention, the blood serum may be further mixed, in the mixing step, with one or more protectant selected from the group consisting of dextran, propylene glycol, sucrose, galactose, triglyceride, and a combination thereof.

Methods of the present invention may be used to protect growth factors in the serum from degradation. The growth factors in the serum include but are not limited to PDGF-AB, TGF-β1, and VEGF.

The present invention is further illustrated by the following examples, which are provided for the purpose of demonstration rather than limitation.

### Example 1: Blood Serum Preparation

Whole blood were collected from volunteer donors must be performed by personal trained in phlebotomy/venipuncture using a double blood bag system (about 50 ml) (TerumoBCT, Japan) with anticoagulant (1 ml of Anticoagulant Citrate Dextrose (ACD) Solution Formula/per 10 ml of blood). After blood collection, gently mix the blood by inverting the tube several times to ensure thorough mixing with anticoagulant. For thorough mixing of blood collected into citrate tubes, it is recommended to invert the tube 3-4 times, while ACD tubes should be inverted eight times. The anticoagulated blood of (A) was activated by adding 1 mL 5 mM of CaCl₂, to generate endogenous thrombin and induce fibrin polymerization and PLT activation. The mixtures were put under mild rotating mixing until clot formation, followed by centrifugation. After centrifugation, a supernatant clear liquid layer (Serum) was present upon a coagulated red blood cell layer. The supernatant liquid (Serum) from either group were pooled, respectively, in sterile-filtered tubes.

### Example 2: Serum Lyophilized Powder Preparation

An appropriate amount of protectants was added to freshly collected serum and mixed thoroughly to obtain a mixture. The mixture was then lyophilized to powder.

TABLE 1
Amount of protectants used glycerol, alginate, and trehalose

| Protectants | Amount |
|---|---|
| Glycerol | 0.01%-10% (v/v) |
| Alginate | 0.01%-10% (w/v) |
| Trehalose | 0.01%-10% (w/v) |
| Albumin | 0.01%-10% (w/v) |
| Triglyceride | 0.01%-10% (v/v) |
| Dextran | 0.01%-10% (w/v) |
| Propylene Glycol | 0.01%-10% (v/v) |
| Galactose | 0.01%-10% (w/v) |
| Sucrose | 0.01%-10% (w/v) |

### Example 3: Growth Factor Level Examination

20 mg serum powder one hour, three month and twelve months after lyophilization, respectively, was dissolved in 1 mL saline and mixed thoroughly. The samples were analyzed within 1 hour after reconstitution by commercially available immunoassays. Standards and samples were assayed in triplicate, and mean values were calculated. The results were multiplied by the dilution factor applied to the samples.

PDGF-AB, TGF-β1, and VEGF levels were measured by ELISA assay.

1. PDGF-AB: PDGF-AB level was assayed using DueSet® ELISA kits (#DY222, R&D Systems, Minneapolis, Minn.). Samples were diluted 20 times in the Reagent Diluent. The plates were incubated for 2 hours, washed, and incubated with enzyme conjugated antibodies to PDGF-AB for an additional 2 hours at room temperature. The wells were washed using the Wash Buffer, then the Substrate Solution was added for 20 minutes at room temperature. Wells were protected from light. Stop Solution was added to each well, and the absorptions at 450 nm were determined using a microplate reader (Gen5, Biotek, VT, USA). The range detectable dose was 15.6-1000 pg/ml.

2. TGF-β1: TGF-β1 level was determined by DueSet® ELISA kits (#DY240, R&D Systems). Samples were diluted 20-fold in the Reagent Diluent. A dilution series of TGF-β1 standards was prepared in 100-μl volumes in 96-well microliter plates coated with TGF-β-receptor II. Before analysis of TGF-β1, acid activation and neutralization was performed to activate latent TGF-β1 to the immunoreactive form. For this purpose, 0.5 ml samples were mixed with 0.1 ml of 1N HCl, incubated at room temperature for 10 minutes, neutralized by an addition of 0.1 ml of 1.2N NaOH/0.5M HEPES (N-[2-hydroxyethyl] piperazine-N0-[2-ethanesulfonic acid]) from Sigma (H-7523), and centrifuged. The supernatant fraction was then assayed for total TGF-β1 content. Aliquots (50 μl) were applied in duplicate to the microliter plate, which was then covered and incubated for 2 h at room temperature. The wells were then washed, enzyme-conjugated polyclonal antibody to TGF-b1 was added, and incubation continued for 2 h at room temperature. Measurements were completed as described above. The range detection limit of TGF-β1 was 31.20-2000 pg/ml.

3. VEGF: VEGF level was assayed using DueSet® ELISA kits (#DY293B, R&D Systems, Minneapolis, Minn.). Samples were diluted 2-fold in Reagent Diluent. The range detectable dose is typically less than 31.2-2000 pg/ml. 100 μl of assay reagent diluent were added to each well, followed by 100 μl of standard (VEGF standard). The plates were covered with adhesives strips and incubated for 2 h at room temperature. The wells were washed 4 times and then incubated with enzyme-conjugated VEGF for 2 h at room temperature. Measurements were completed as described above.

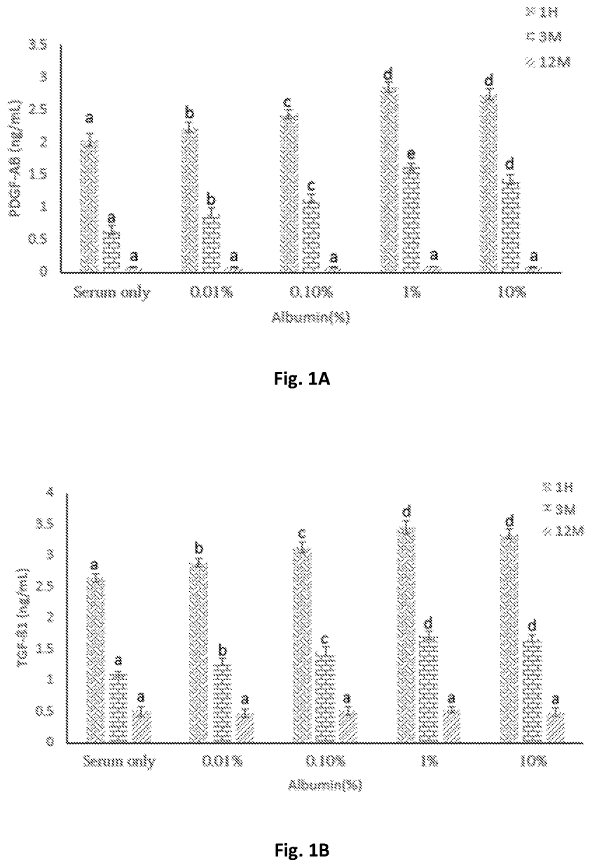

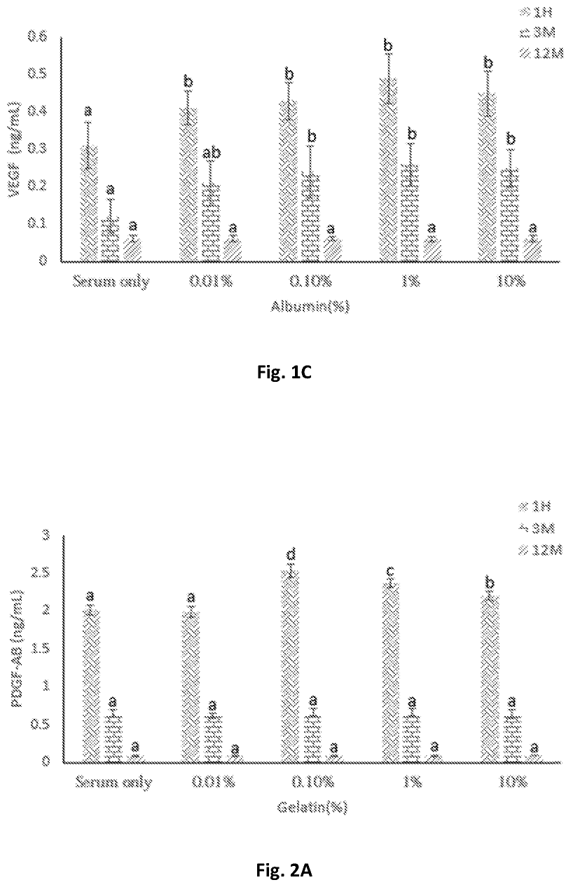

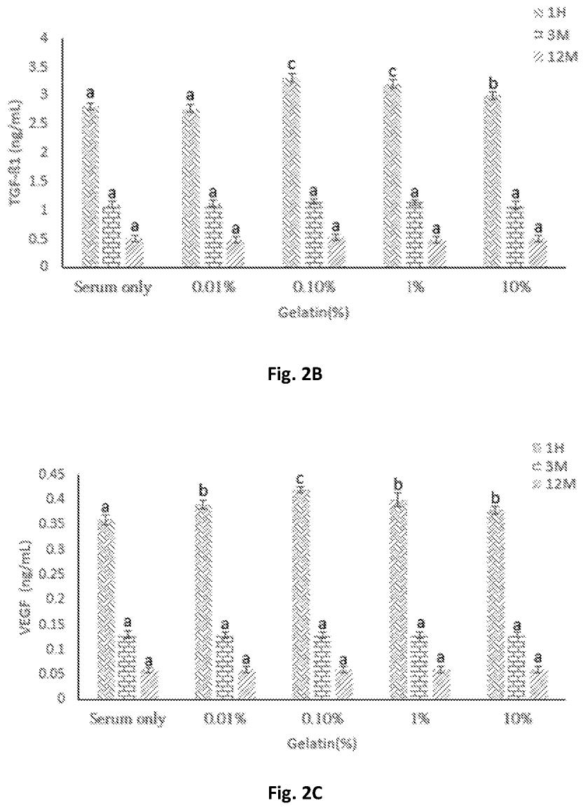

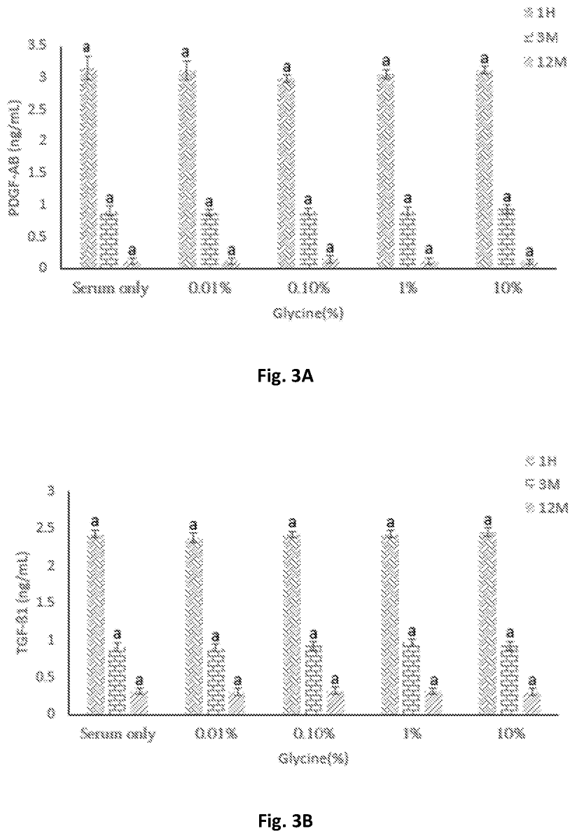

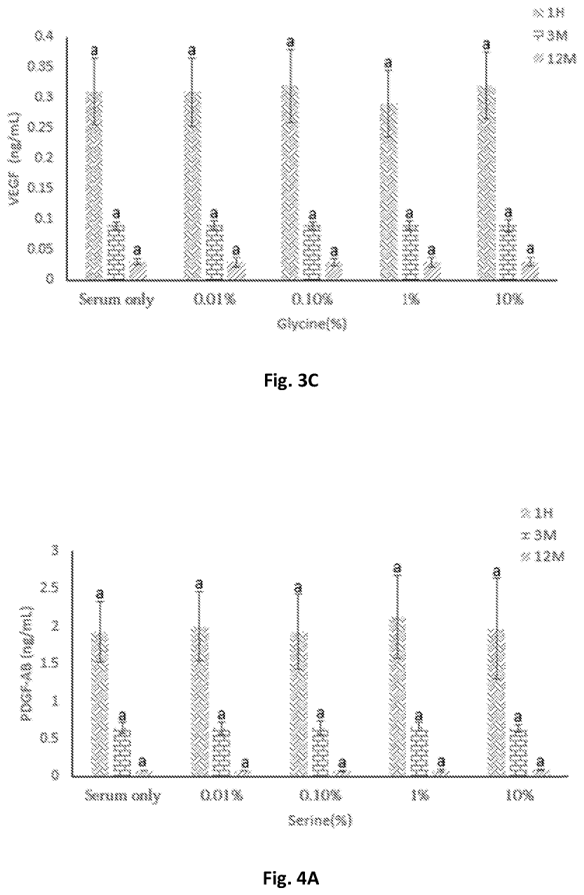

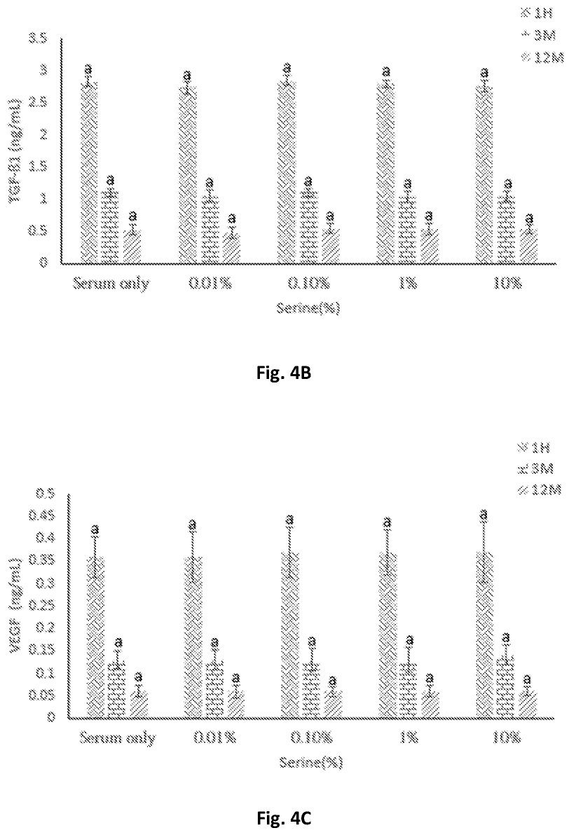

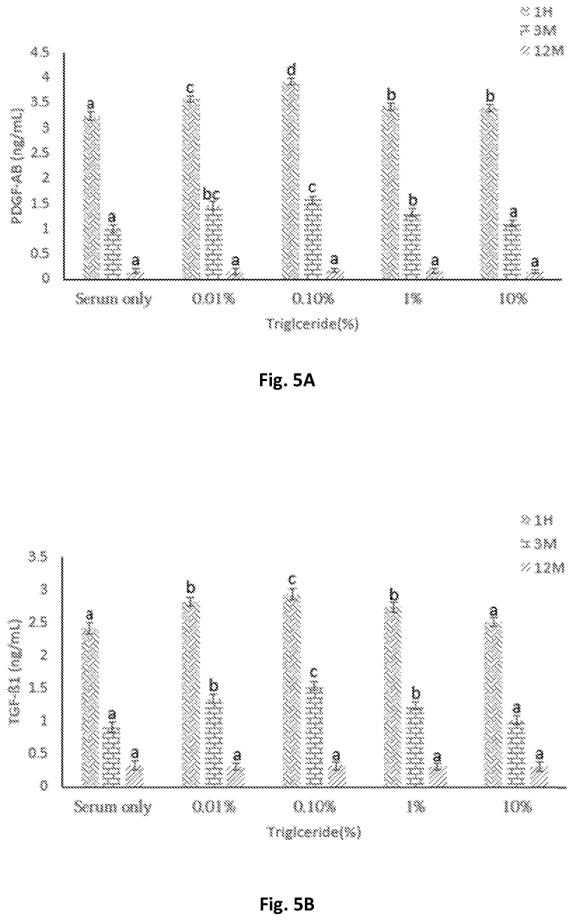

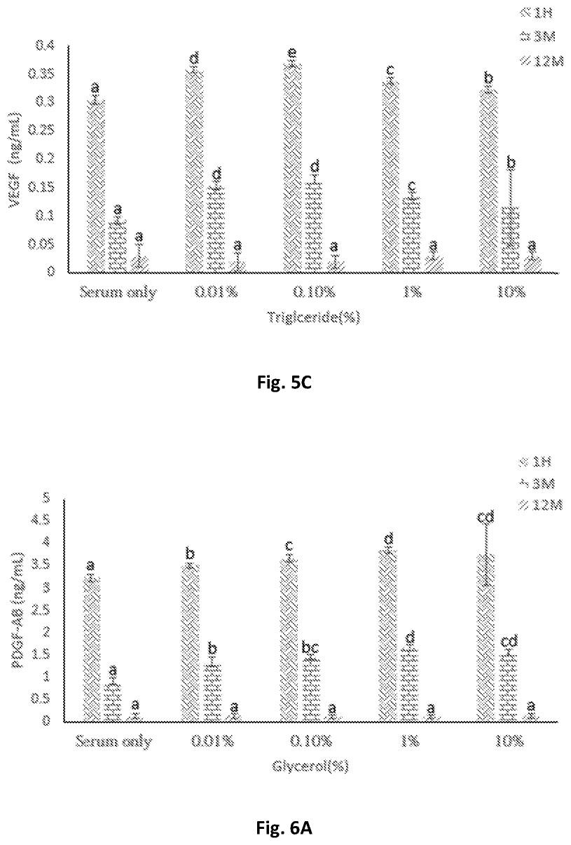

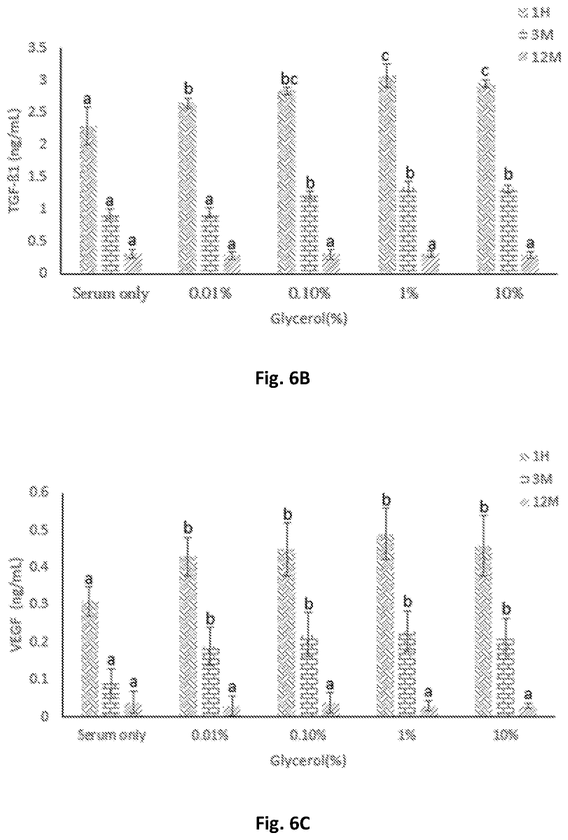

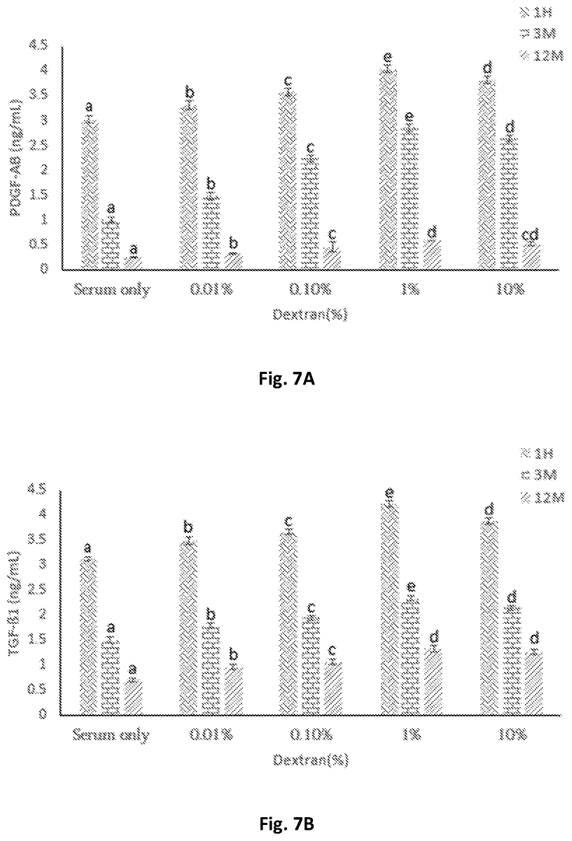

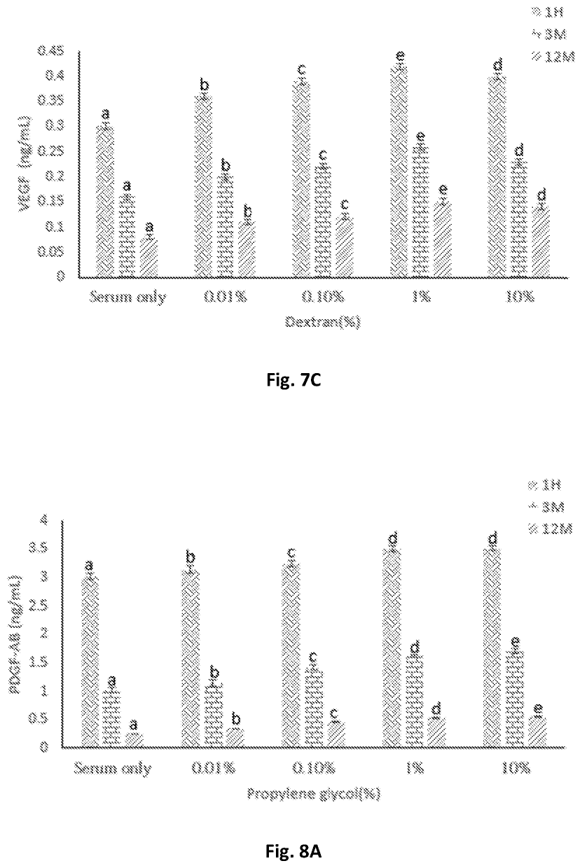

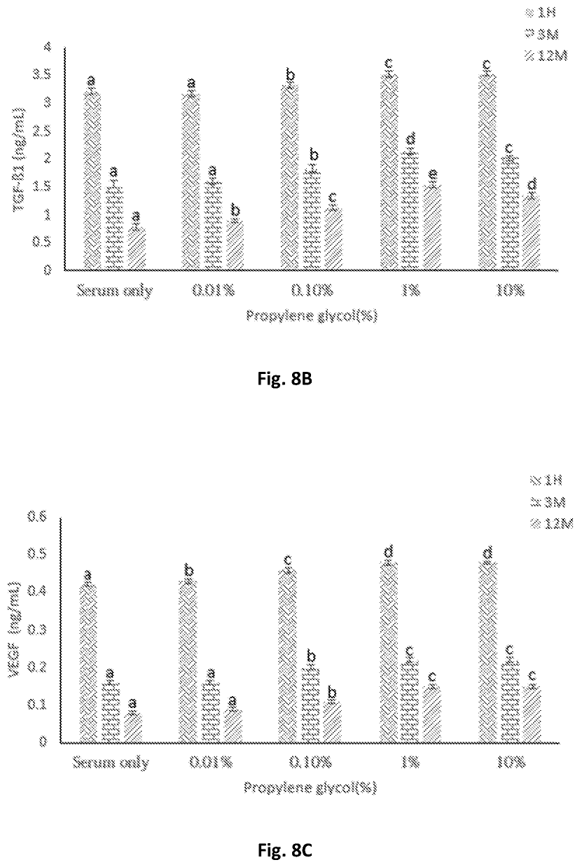

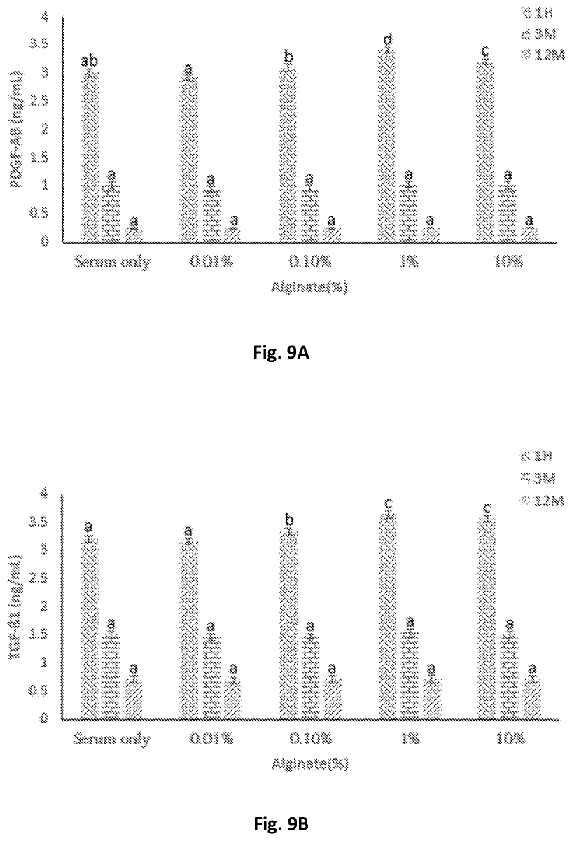

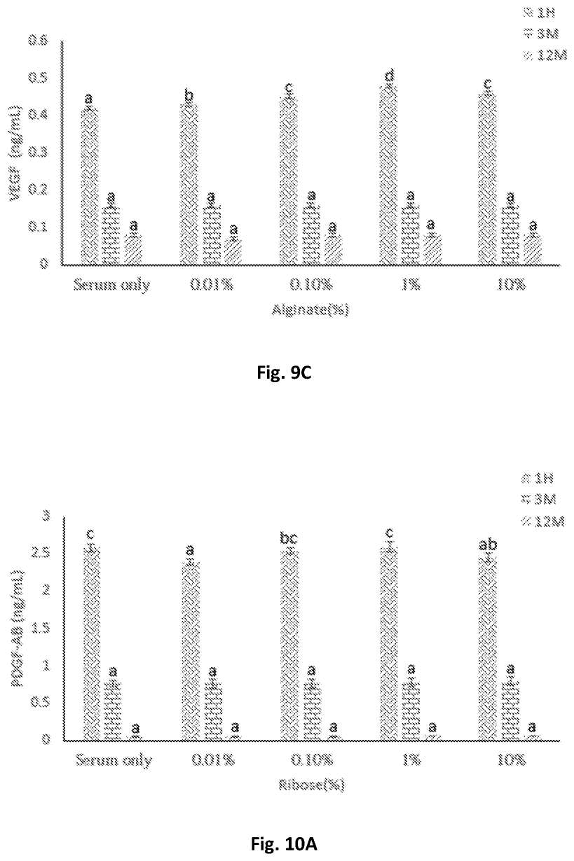

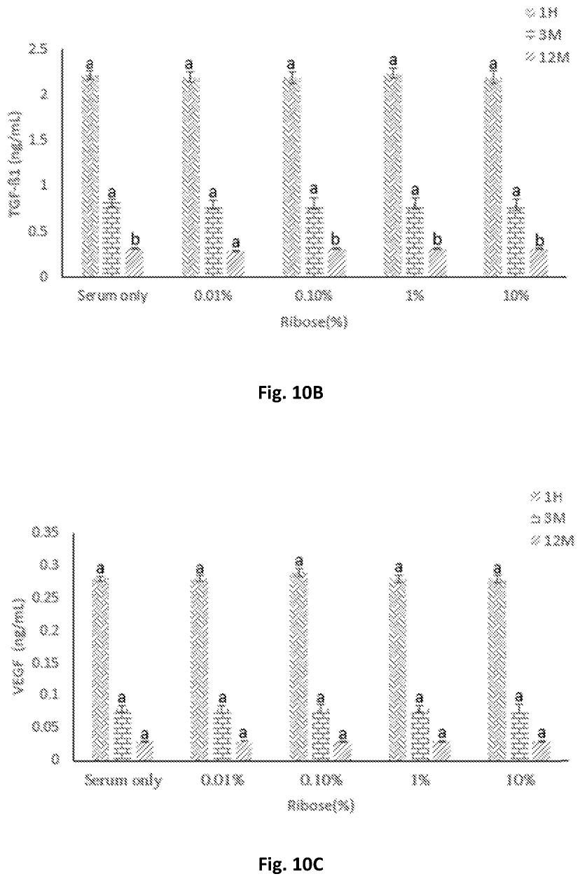

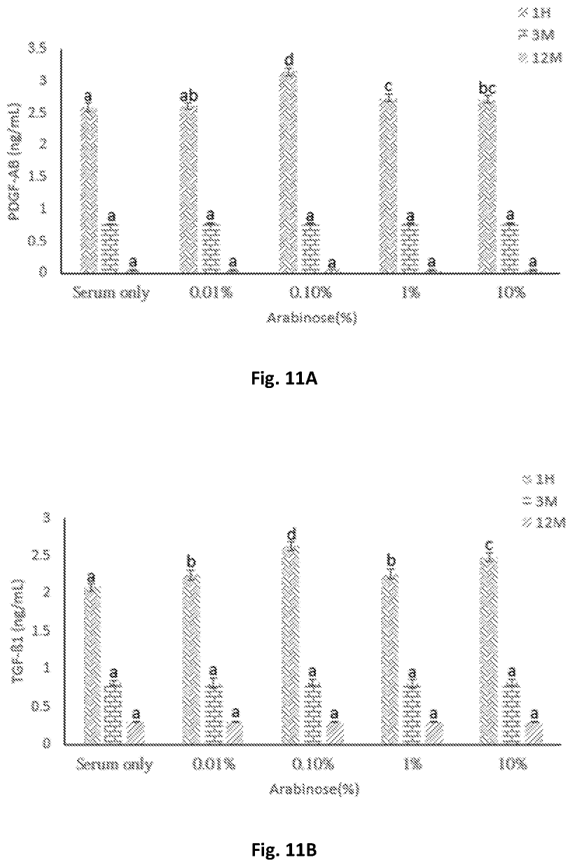

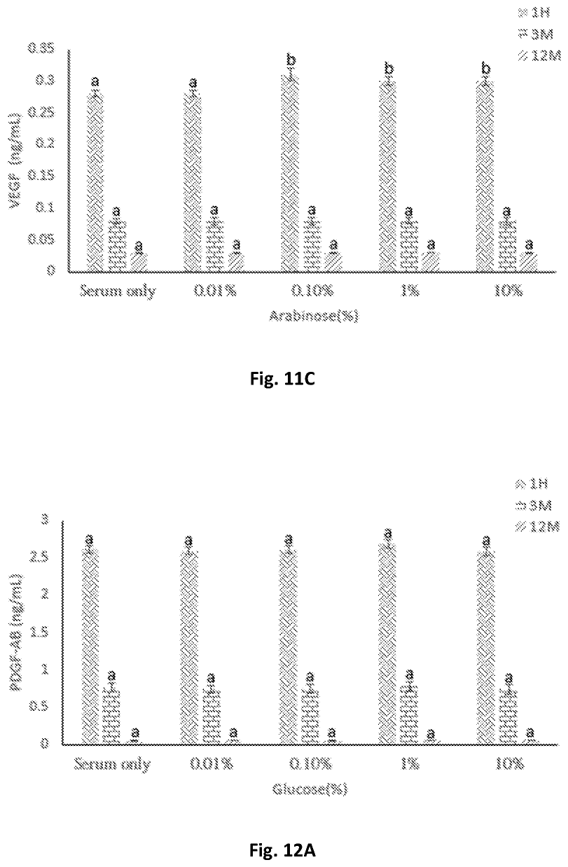

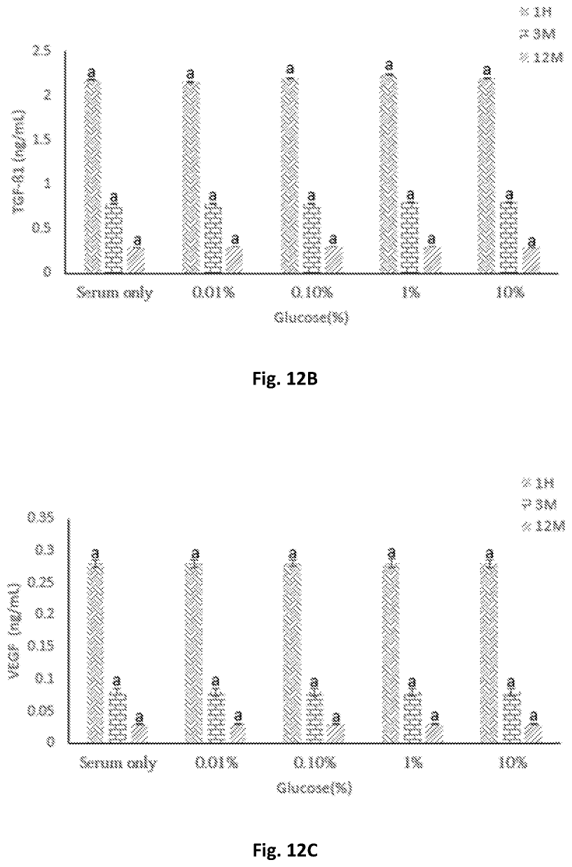

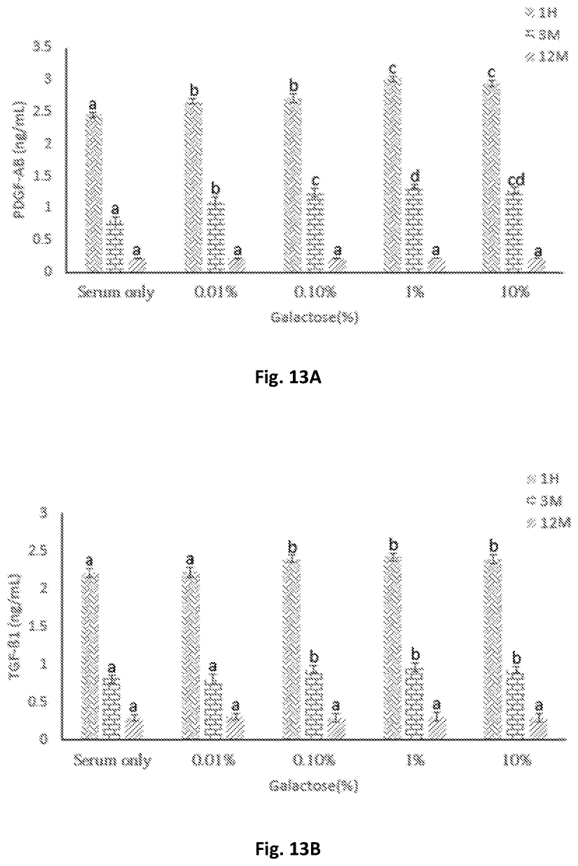

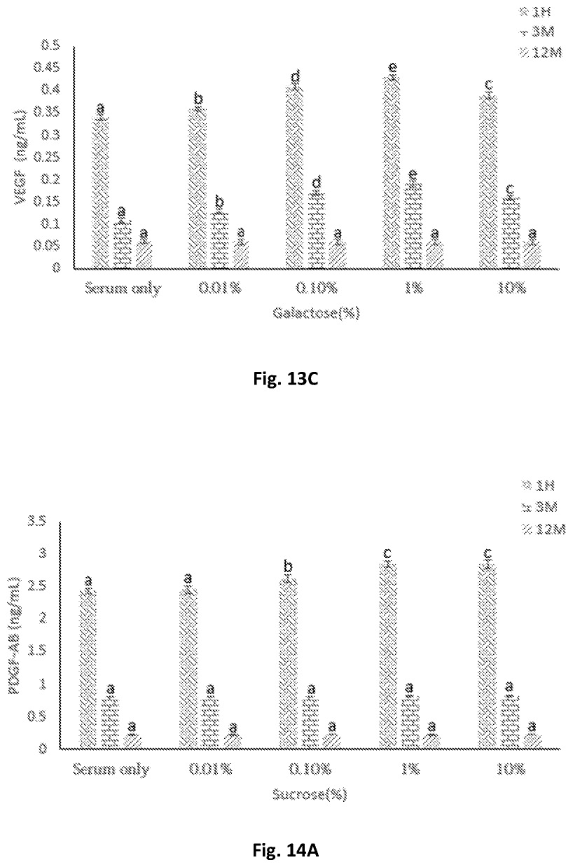

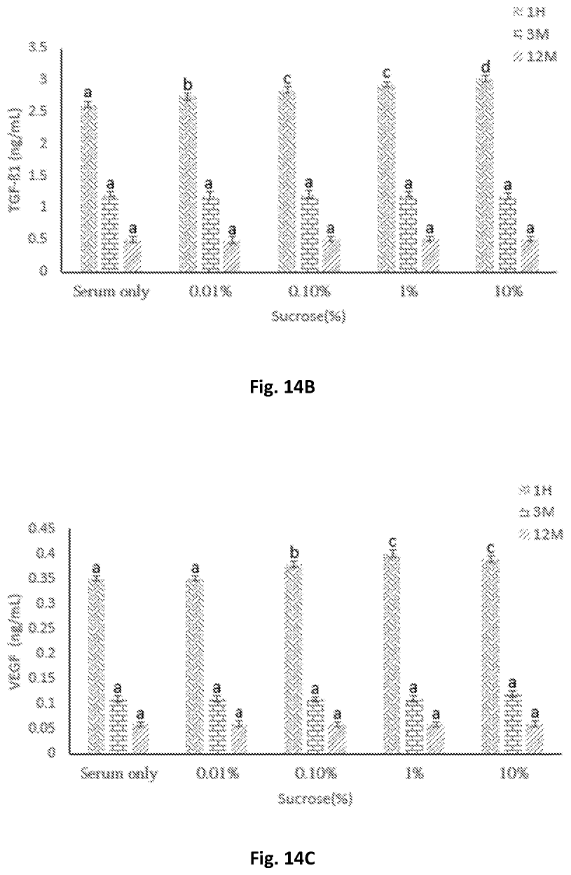

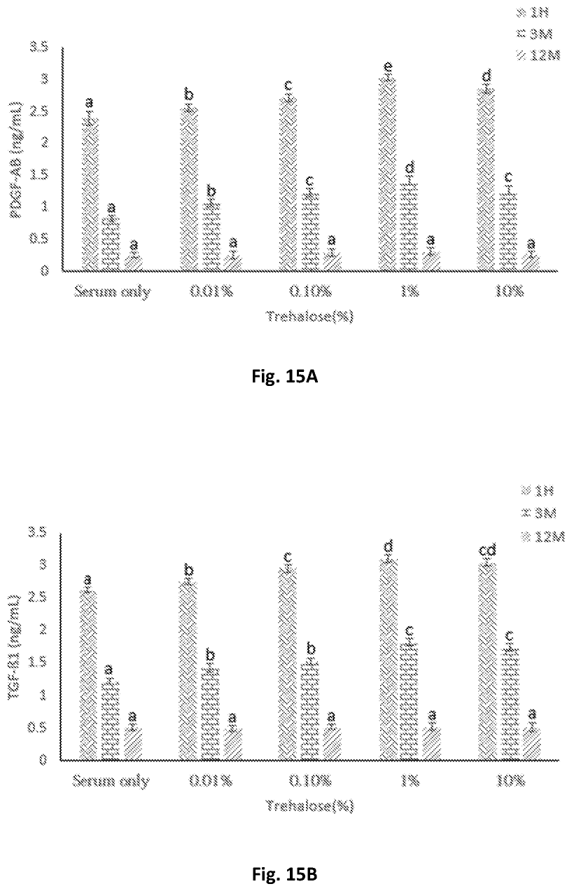

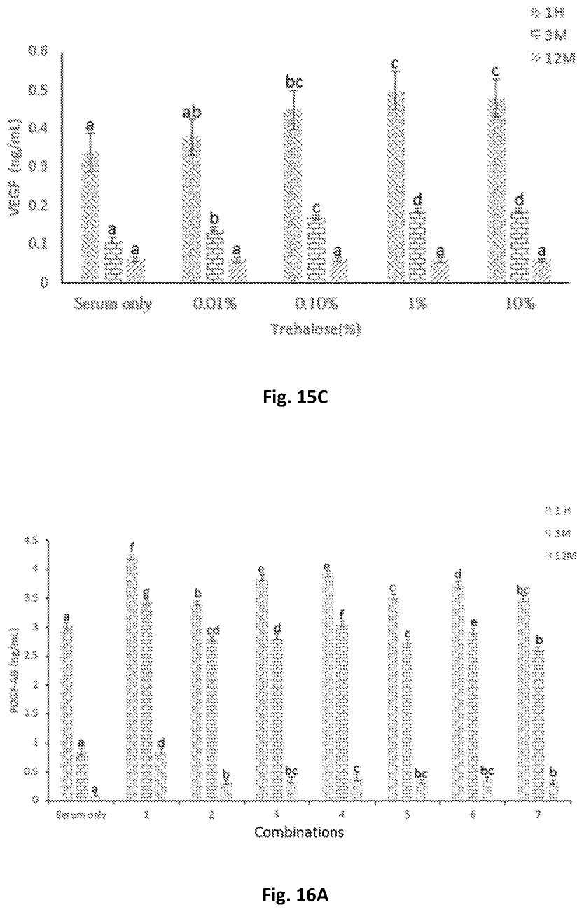

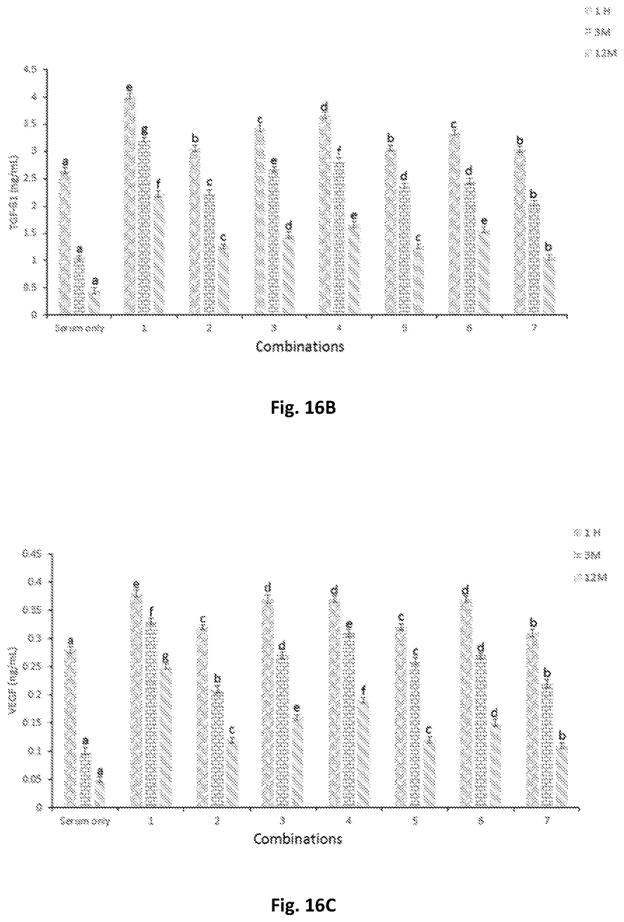

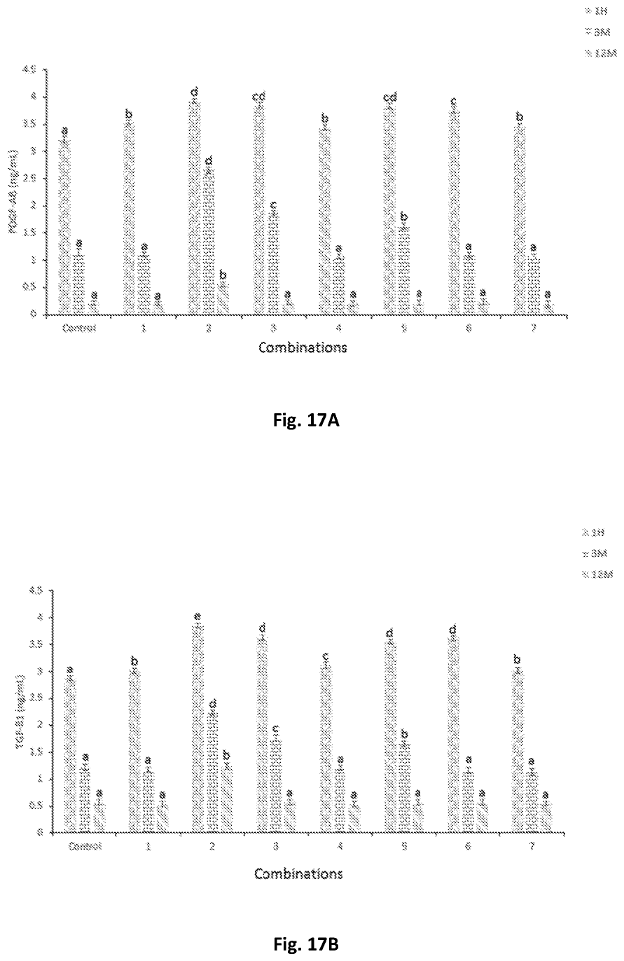

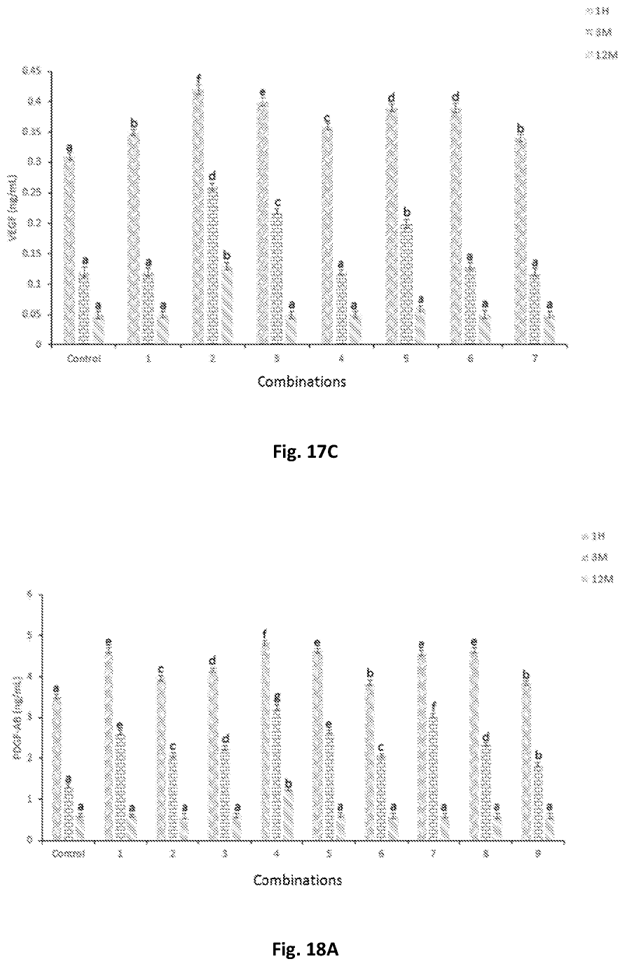

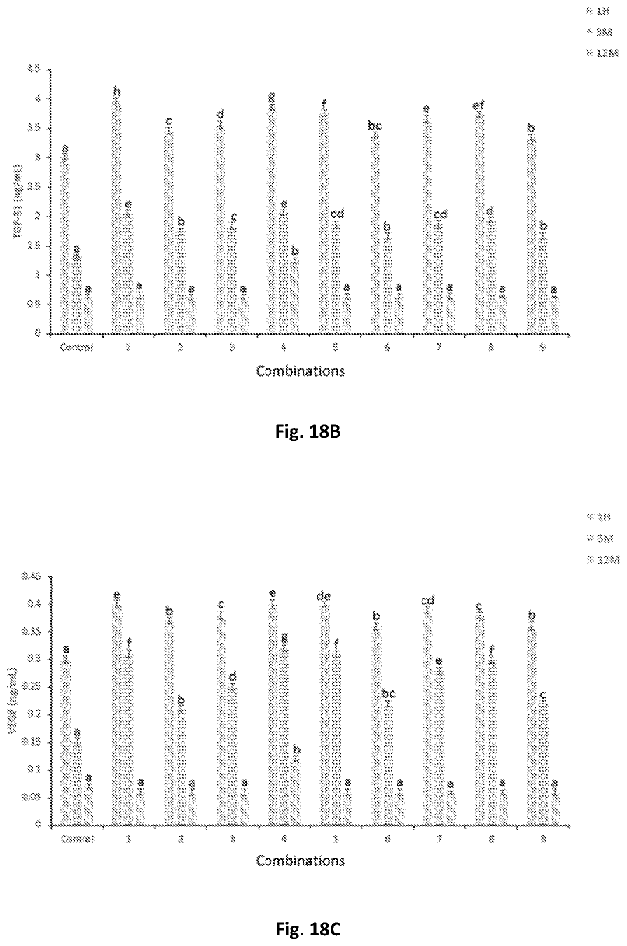

All tests were repeated three times, and the results were analyzed by one-way ANOVA, F-test and Duncan test by SPSS22 software, and expressed as Mean±SD. Means in the same bar stripe of storage time with different letters are significantly different (P<0.05). The results are shown in FIGS. 1A-18C.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for blood serum protein activity preservation, comprising mixing blood serum with one or more protectants selected from the group consisting of albumin, triglyceride, glycerol, dextran, propylene glycol, galactose, alginate, and trehalose to obtain a mixture; and lyophilizing the mixture.

2. The method of claim 1, comprising mixing blood serum with two or more protectants selected from the group consisting of albumin, triglyceride, glycerol, dextran, propylene glycol, galactose, alginate, and trehalose to obtain a mixture; and lyophilizing the mixture.

3. The method of claim 2, wherein the two or more protectants are selected from the group consisting of glycerol, alginate, and trehalose.

4. The method of claim 3, wherein the two or more protectants comprise a first protectant of trehalose and a second protectant of glycerol or alginate.

5. The method of claim 4, wherein the two or more protectants comprise trehalose and glycerol.

6. The method of claim 4, wherein the two or more protectants comprise trehalose and alginate.

7. The method of claim 3, wherein in the mixing step the blood serum is further mixed with one or more protectant selected from the group consisting of dextran, propylene glycol, sucrose, galactose, triglyceride, and a combination thereof.

8. The method of claim 4, wherein in the mixing step the blood serum is further mixed with one or more protectant selected from the group consisting of dextran, propylene glycol, sucrose, galactose, triglyceride, and a combination thereof.

9. The method of claim 5, wherein in the mixing step the blood serum is further mixed with one or more protectant selected from the group consisting of dextran, propylene glycol, sucrose, galactose, triglyceride, and a combination thereof.

10. The method of claim 6, wherein in the mixing step the blood serum is further mixed with one or more protectant selected from the group consisting of dextran, propylene glycol, sucrose, galactose, triglyceride, and a combination thereof.
